(19) United States
(12) Patent Application Publication
(10) Pub. No.: US 2005/0130185 A1
(43) Pub. Date: Jun. 16, 2005

(54) Sepsis detection chip and fabrication method thereof and method of detecting sepsis

(76) Inventors: Kun-Shan Lu, Taipei (TW); Cheng-Yu Wei, Hualien City (TW); Yu-Jie Zhao, Shenyang City (CN)

Correspondence Address: J C PATENTS, INC., 4 VENTURE, SUITE 250, IRVINE, CA, 92618, US

(21) Appl. No.: 10/911,164

(22) Filed: Aug. 3, 2004

(30) Foreign Application Priority Data
Dec. 12, 2003 (TW) 92135132

(51) Int. Cl. C12Q 1/68; C12M 1/34

(52) U.S. Cl. 435/6; 435/287.2; 427/2.11

(57) ABSTRACT

A sepsis microarray detection chip includes a plurality of probes immobilized on a matrix, wherein each probe is selected from the group of deoxyribonucleotide sequences depicted in the SEQ ID NOs. 1 to 66. Since these probes are formed with deoxyribonucleotide sequences specific to sepsis, they can be used to detect sepsis.

### CROSS-REFERENCE TO RELATED APPLICATION

This application claims the priority benefit of Taiwan application serial no. 92135132, filed on Dec. 12, 2003.

### BACKGROUND OF THE INVENTION

1. Field of the Invention

The present invention relates to a detection device for an illness, a fabrication method thereof and a method for detecting the illness. More particularly, the present invention relates to a sepsis detection chip, a fabrication method thereof and a method for detecting sepsis.

2. Description of Related Art

Sepsis is a common disease. Although the morbidity of sepsis becomes lower as the medicine technologies keep progressing, the onset of serious sepsis can still be fetal. Therefore, rapid and accurate diagnosis is crucial to the treatment of sepsis. The treatments of sepsis in fact vary depending on the pathogens, the immunity of the patients and the medical resources available. Due to the abuse of antibiotics, different pathogens may exhibit diverse drug resistance, distinct pathogenicity and toxicity, which makes the treatment of sepsis more difficult.

Conventionally, methods for verifying the pathogens of sepsis include the Gram's stains, antibody based immunology tests and the bacteria culture etc. Though the microscopic examination of the Gram's stains is a simple and fast way to analyze the pathogens of sepsis, it requires relatively large amounts of bacteria for the examination. While the immunology test is faster than the bacteria culture, it is inefficient because the sample is only tested for a single antibody one time. The required time for the bacteria culture is the major obstacle, despite of a higher sensitivity. Moreover, application of antibiotics could result in pseudo-negative results of the Gram's stain or the bacteria culture, thus misleading the clinical practitioners.

Microarray detection chip is one of the most important technological advancements in the high-tech field in recent years. Microarray detection chip is a high technology product that bases on a multidisciplinary effort from areas of physics, chemistry, microelectronics, precision machining and bioscience. Mircorarray detection chip can provide a large amount of probes with specific DNA sequences immobilized on a matrix, and a great deal of information is produced after the probe is reacted with a test sample (for example, DNA). Therefore, a microarray detection chip can be used for screening diseases. However, for different types of diseases, a great deal of effort must be devoted to the fabrication of the microarray detection chip in order to design a specific probe and a specific primer set (pair) to amplify the DNA fragment of the test sample of the patient. Currently, no such microarray detection chip has been developed for screening sepsis.

### SUMMARY OF THE INVENTION

Accordingly, the present invention provides a fabrication method for a sepsis detection chip, wherein the resulting detection chip can concurrently screen a plurality of sepsis pathogens.

In accordance to the present invention, a microarray detection chip for sepsis is provided, wherein prior art problems of inaccurate test results are resolved.

The present invention further provides a detection method for sepsis for accurately and rapidly detecting whether a patient has contracted sepsis and the pathogen type of sepsis.

The present invention provides a fabrication method for a sepsis detection chip, wherein the method includes designing a plurality of probe sequences and these probe sequences includes at least the DNA sequences depicted in SEQ ID NO. (Sequence Identifier Number) 1 to SEQ ID NO. 66. The design of the probe sequences further includes designing a primer set that corresponds to the probe sequences for amplifying specific DNA fragments of the test sample from the patient. The primer set is formed with the DNA sequence depicted in the SEQ ID NOs. 67 and 68. The probe synthesis step is conducted to synthesize the probes including DNA sequences depicted in SEQ ID NO. 1 to SEQ ID NO. 66. Thereafter, a spotting step is performed to spot respectively these probes onto a matrix.

The present invention provides a microarray detection chip, wherein the detection chip is applicable for detecting whether a patient has contracted sepsis. This detection chip includes immobilizing a plurality of probes on a matrix, wherein each probe is selected from the group consisting of the DNA sequences depicted in the SEQ ID NOs. 1-66.

The present invention provides a detection method for sepsis, wherein this method is applicable for detecting whether a patient has sepsis. This detection method includes providing the aforementioned microarray detection chip. After treating the sample from the patient and obtaining a DNA from the sample, a primer set is used to conduct a polymerase chain reaction (PCR) to amplify a specific DNA fragment and to obtain a PCR product. The primer set used in the PCR is formed with the DNA sequences depicted in the SEQ ID NOs. 67 and 68. Then, a hybridization procedure is conducted so that this PCR product reacts with the probes on the microarray detection chip. Thereafter, the test result from the microarray detection chip is analyzed.

Since the microarray detection chip of the present invention employs the DNA sequence specific to sepsis, this detection chip can be used to detect whether a patient has sepsis and the type of sepsis.

Since the DNA sequences specific to the pathogens of sepsis are used as the probes in the microarray detection chip, the microarray detection chip of this invention can be used to examine whether the patient has sepsis and to detect the pathogen type of sepsis.

It is to be understood that both the foregoing general description and the following detailed description are exemplary, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention.

FIG. 1 is a flow diagram illustrating the fabrication method for a sepsis detection chip according to one embodiment of the invention.

FIG. 2 is a flow diagram illustrating the method of detecting sepsis according to one embodiment of the invention.

### DESCRIPTION OF THE EMBODIMENTS

The important aspects in the fabrication of a microarray detection chip are the designs of the probes and the primers set for amplifying the DNA segments of a patient's sample. The microarray detection chip can provide accurate information only with the design of specific probes and specific primer sets. The fabrication method of the microarray detection chip for sepsis and the related detection method provided in the present invention are based on the aforementioned concepts. Although the disclosure herein refers to certain illustrated embodiments, it is to be understood that these embodiments are presented by way of example and not by way of limitation.

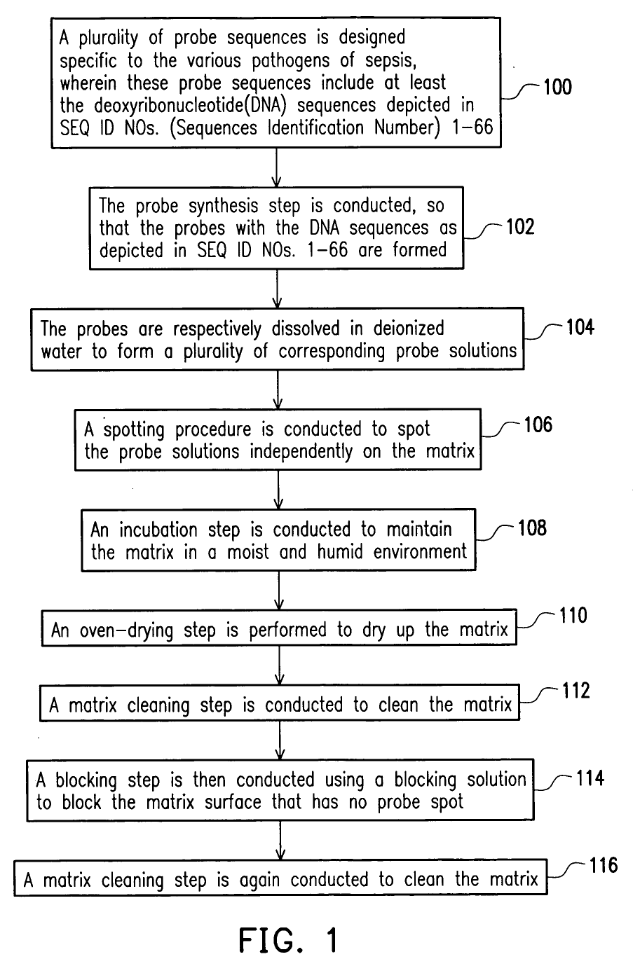

FIG. 1 is a flow diagram illustrating the fabrication method of a sepsis detection chip according to one embodiment of the invention.

Referring to FIG. 1, a plurality of probe sequences is designed specific to the various pathogens of sepsis, wherein these probe sequences include at least the deoxyribonucleotide (DNA) sequences depicted in SEQ ID NOs. (Sequence Identification Number) 1-66 (step 100). These probe sequences, for example, are formed with 15 to 30 deoxyribonucleic acids. These probe sequences are designed according to the available gene sequences of the pathogens of sepsis obtained from the gene bank.

Table 1 lists the pathogens of sepsis and the corresponding SEQ ID NOs. of the probe sequences.

TABLE 1

| | SEQ ID Nos. of the probe |
|---|---|
| Pathogens | sequences |
| Acimetobacter | 1 |
| Actinomyces | 2 |
| Borrelia burgdorfri | 3 |
| Burkholderia cepacia | 4 |
| Bacteroides fragilis | 5 |
| Burkholderia pseudomallei | 6 |
| Bartonella sp. | 7 |
| Borrelia afzelii | 8 |
| Borrellia garinii | 9 |
| Clostridium difficile | 10 |
| Clostridium perfringens | 11 |
| Capnocytophaga ochracea | 12 |
| Chlamydia pneumoniae | 13 |
| Clostridium septicum | 14, 15 |
| Coxiella burnetii strain 1 M | 16 |
| Escherichia coli | 17 |
| Enterobacter aerogenes | 18 |
| Enterococcus dispar[dyran] | 19, 20 |
| Enterococcus faecalis straink4 | 21, 22 |
| Enterococcus faecium | 23 |
| Flavimonas oryzihabitans | 24 |
| Flavobacterium sp. | 25 |
| Fusobacterium necrophorum | 26 |
| Haemophilus influenzae | 27 |
| Klebsiella oxytoca-16 | 28, 29 |
| Klebsiella pneumoniae | 30 |
| Leptospira interrogans | 31 |
| Lactobacillus sp. B5406 | 32 |
| Lgionella sp. Strain LLAP9 | 33 |
| Mycobacterium tuberculosis | 34 |
| Moraxella sp. | 35 |
| Mycoplasma pneumonia strain ACTT15531 | 36 |
| Neisseria meningitides | 37 |
| Nocardia asteroids | 38 |
| P-mor | 39 |
| P-pro | 40 |
| Petococcus niger | 41 |
| Petostreptococcus sp. | 42 |
| Prevotella intermedia ATCC25611 | 43 |
| Proteus vulgaris | 44 |
| Pseudomonas sp. Strain Circle | 45 |
| Rhodococcus sp | 46 |
| Rickettsia typhi strain Wilmington | 47 |
| Staphylococcus epidermidis | 48, 49 |
| Staphylococcus haemolyticus | 50, 51 |
| s-aea | 52 |
| Streptococcus agalactiae | 53 |
| Streptococcus bovis | 54 |
| Streptococcus milleri | 55 |
| Streptococcus pyogenes | 56 |
| Staphylococcus haemolyticus | 57 |
| Salmonella | 58 |
| Serratia sp. | 59 |
| Staphylococcus aureus, Staphylococcus | 60 |
| saccharolyticus | |
| Stenotrophomonas sp. | 61 |
| Streptococcus sp. | 62 |
| Treponema pallidum | 63 |
| V. cholerae | 64 |
| V. vulnificus | 65 |
| Bacillus subtilis | 66 |

It is noted that the pathogen Clostridium septicum should be discriminated by both probe sequences of the SEQ ID NOs. 14 and 15 in the following test procedure. That is, the pathogen of sepsis is identified Clostridium septicum only if both probe sequences of the SEQ ID NOs. 14 and 15 show positive results. Similarly, the pathogen Enterococcus dispar[dyran] should be identified by both probe sequences of the SEQ ID NOs. 19 and 20. The pathogen Enterococcus faecalis straink4 should be identified by both probe sequences of the SEQ ID NOs.21 and 22. The pathogen Klebsiella oxytoca-16 should be identified by both probe sequences of the SEQ ID NOs. 28 and 29. The pathogen Staphylococcus epidermidis should be identified by both probe sequences of the SEQ ID NOs. 48 and 49, while the pathogen Staphylococcus haemolyticus should be identified by both probe sequences of the SEQ ID NOs. 50 and 51. Moreover, the probe sequence of the SEQ ID NO. 60 is basis for detecting the pathogens Staphylococcus aureus and Staphylococcus saccharolyticus.

The step of designing the probe sequence (step 100) includes designing the primer set that corresponds to these probe sequences, which is used to amplify the specific DNA sequence of the patient's sample. According to one preferred embodiment, the primer set includes the common primer set formed from the DNA sequences depicted in the SEQ ID NOs. 67 and 68. That is, the specific DNA fragment of the diseased pathogens listed in Table 1 can be amplified by using this primer set. Similarly, the design of these primer set is based on the reliable gene sequences of the pathogens of sepsis obtained from a gene bank.

Each primer set includes a 5′ to 3′ forward primer and a 3′ to 5′ reverse primer. According to one preferred embodiment, the forward primer is the DNA sequence of SEQ ID NO. 67, while the reverse primer is the DNA sequence of SEQ ID NO. 68. The primer set is used to amplify the specific DNA fragment (16s) of the pathogens of sepsis.

Then, performing the step of synthesizing the probes (step 102), so that the probes with the DNA sequences as depicted in SEQ ID NOs. 1-66 are formed. Further, during the probe synthesis step (step 102), the 5′ ends of the DNA sequences depicted in the SEQ ID NOs. 1-66 are also modified. Consequently, these probes can covalently bind with the functional groups on the matrix surface to be immobilized on the matrix surface. For example, the modification includes adding an amino group to the 5′ end of the probe.

Thereafter, the synthesized probes are dissolved in deionized water to form a plurality of probe solutions (step 104). The probe solution has a concentration of, for example, 200 mmol/L.

A spotting procedure (step 106) is then conducted to spot the probe solutions independently on the matrix. Depending on the number of spots required, the radius of the spots is about of 50 to 300 microns. Further, depending on the situation, each type of probe solutions can be spotted more than once. At least 66 spots of the probe solutions are formed on the matrix. The surface area of the matrix is sufficiently large to accommodate tens to thousands of spots. The material of the matrix is, for example, glass. Due to the amino modification to the 5′ end of the DNA sequences in the previous probe synthesis step (step 102), the probe solution is spotted on the matrix and is immobilized on the matrix through covalent bonding.

Thereafter, an incubation step is conducted to maintain the matrix in a moist and humid environment (step 108). The incubation step is conducted at 37 degrees Celsius for three days continuously, for example.

An oven-drying step is performed to dry up the matrix (step 110). This oven-drying step is conducted at 80 degrees Celsius for 2 hours, for example.

A matrix cleaning step is then conducted to clean the matrix (step 112), wherein the matrix cleaning step includes performing a cleaning process and a drying process. The cleaning solution used in the cleaning process consists of a probe buffer solution and deionized water. The probe buffer soltiion is formed with 1×SSC and 0.1% of sodium dodecyl sulfate (SDS), while SSC is a solution with a pH of about 7 and is formed with 3M of NaCl and 0.3M of sodium citrate. The drying process is accomplished by blowing drying the matrix using nitrogen gas.

A blocking step is then conducted using a blocking solution to block the matrix surface that has no probe spots (step 114). The blocking solution used is, for example, a pH 7 solution formed with 1% bovine serum albumin (BSA) and 0.01 mol/L of phosphate buffer (PB).

A matrix cleaning step (step 116) is again conducted to clean the matrix. This matrix cleaning step includes performing a cleaning process, followed by a drying process. Further, this matrix cleaning step 116 can be repeated for several times until the matrix is completely cleaned. The cleaning solution used in this cleaning step includes, for example, deionized water for cleaning the excess blocking solution. The drying process is, for example, using nitrogen gas to blow dry the matrix. In one preferred embodiment, the matrix cleaning step is repeated, for example, for three times.

The fabrication of the detection chip is completed with the aforementioned method. The detection chip comprises a plurality of specific DNA sequences related to sepsis. Therefore, various pathogens of sepsis can be detected concurrently by using the detection chip.

It is also worth noting that during the step of designing the probes (step 100), a plurality of quality control probe sequences can also design. These quality control probes are synthesized and immobilized on the matrix through the probe synthesis step and the spotting step (steps 102 to 116). The quality control probes are related to the sequence of a specific substance in the test sample, which are used to ensure the sample extracted is an effective sample to prevent misjudgment of the test result.

Further, using the above method, the detection chip obtained includes a plurality of probes immobilized on the matrix. Further, each probe is selected from the group consisting of the DNA sequences depicted in the SEQ ID NOs. 1-66. Further, each probe is formed with 15 to 30 deoxyribonucleic acids. The material of the matrix is, for example, glass.

In another embodiment, each DNA sequence depicted in the SEQ ID NOs. 1-66 is immobilized on the matrix, wherein these DNA sequences are used as probes to detect the pathogens of sepsis. Further, the matrix is not only disposed with these 66 probes. Depending on the situation required, the sequences recited in SEQ ID NOs. 1-66 can be repeated for several times to constitute a microarray detection chip with tens or several thousands probes.

Since these probe sequences are related to the gene sequences of various pathogens of sepsis, they can be used to determine whether the patient has contracted sepsis and the type of sepsis.

The method for detecting whether a patient has contracted sepsis using the microarray detection chip that is fabricated according to the above method, is detailed in the following.

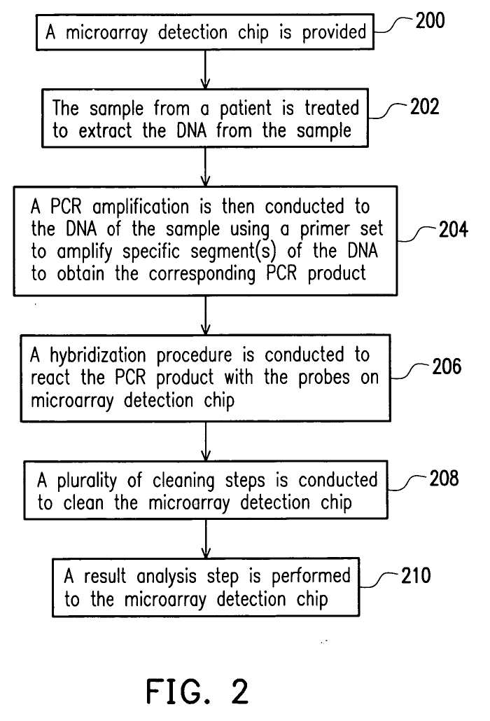

FIG. 2 is a flow diagram illustrating a method for detecting sepsis according to one embodiment of the present invention.

Referring to FIG. 2, a microarray detection chip is provided (step 200), wherein this microarray detection chip is fabricated using, for example, the aforementioned method. Further, this microarray detection chip includes a plurality of probes specific for detecting the pathogens of sepsis. In one embodiment of the invention, besides these specific probes for detecting the pathogens of sepsis, this microarray detection chip further includes quality control probes immobilized thereon.

Thereafter, the sample from a patient is treated to extract the DNA from the sample (step 202), wherein the sample from the patient is, for example, blood. For example, the blood sample is the venous blood from the patient infected by Staphylococcus Aureus. The treatment includes extracting the obtained 3-5 ml venous blood by using phenol-chloroform and followed by precipitating with ethanol to obtain the DNA. If the DNA needs to be stored for a longer period time, it can be preserved at −20 degrees Celsius.

A PCR amplification is then conducted to the DNA of the sample using a primer set to amplify specific segment(s) of the DNA to obtain the corresponding PCR product (step 204). The primer set used in the PCR amplification is the primer set formed with the DNA sequences depicted in the SEQ ID NOs. 67 and 68.

The reagent used in the PCR amplification includes at least the DNA from the sample, DNA polymerase, the above primer set and deoxyribonucleoside triphosphate (dNTP), wherein the DNA polymerase is, for example, Tag enzyme. Further, the PCR product is a PCR product labeled with a label. The method for labeling the PCR product includes, for example, using a labeled primer set, a labeled deoxyuridine triphosphate (dUTP) or a labeled dNTP and the above reagent to perform the PCR amplification. These labels are, for example, Cy3, Cy5 or other appropriate fluorescent materials. The label of the PCR product serves as a reference in the subsequent analysis process for determining whether the PCR product has reacted with the probe.

In the above PCR procedure, symmetric or asymmetric multiplex polymerase chain reaction is conducted. In other words, the concentration of the forward primers and the reverse primers can be different to produce single stranded DNA in the PCR product. The single stranded DNA is subsequently hybridized with the single stranded probe on the microarray detection chip. If the concentration of the forward primer sets and the reverse primer sets are the same, a thermal denaturation procedure is required before the hybridization process to separate the two complementary single DNA strands before proceeding with the hybridization procedure.

The conditions for PCR amplification are shown as follow in Table 2.

| STEP | Reaction Temperature | Duration Period (sec.) |
|---|---|---|
| 1 | 94 | 5 |
| 2 | 94 | 0.5 |
| 3 | 56 | 0.5 |
| 4 | 72 | 1 |
| 5 | 72 | 5 |

In this embodiment, the PCR amplification is conducted according to the conditions in step 1, followed by repeating the conditions in steps 2 to 4 for 30 cycles, and is concluded according to the conditions in step 5.

Thereafter, a hybridization procedure is conducted to react the PCR product with the probes on microarray detection chip (step 206). The hybridization reaction is conducted, for example, in an environment of about 60 degrees Celsius for about 2 hours. Further, the hybridization reaction is conducted by, for example, using a hybridization buffer, wherein the amount of the hybridization buffer used is the same as the amount of the PCR product. The hybridization buffer consists of 10×SSC and 0.1% SDS. In this procedure, if the sequence of the PCR product with the single stranded structure is complementary to the sequence of the probe, the PCR product is hybridized with the probe. Further, since the PCR product comprises a label, the label can be used to identify the type of probe that is hybridized with the PCR product in a subsequent process.

A plurality of cleaning steps is conducted to clean the microarray detection chip (step 208), wherein the cleaning solution used in these cleaning steps is, for example deionized water. In one preferred embodiment, the microarray detection chip is repeatedly cleaned for three times. Further, with these cleaning procedures (step 208), the PCR product that has not been hybridized with the probe is washed out, leaving only the PCR product that is complementary to the probe.

Thereafter, a result analysis step is performed to the microarray detection chip (step 210). The result analysis step includes performing a scanning procedure and a data analysis procedure, wherein the scanning procedure includes, for example, using a scanner to scan the mircroarray detection chip to obtain information on various test results. Further, the data analysis procedure includes, for example, using an analysis software that is compatible with the scanner to output the analysis results of the microarray detection chip. Since the PCR product after hybridization is labeled, the scanner can identify whether a label is present at the position of each probe (for example, an emission of a fluorescent signal) during the scanning procedure with the scanner. Therefore, based on the data analysis procedure, the pathogen of sepsis contracted by the patient is identified. If the microarray detection chip shows no label at all besides the quality control probes, it is an indication that the patient does not have sepsis.

In accordance to the present invention, the fabrication method of a sepsis detection chip is provided, wherein this detection chip includes a plurality of deoxyribonucleotide sequences specific to the DNA sequences related to various types of sepsis. Therefore, the detection chip is applicable to determine whether the patient has contracted sepsis and the type of sepsis.

By using the microarray detection chip of this invention for detecting sepsis, a large amount of accurate analysis results are attained.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

1. A fabrication method of a sepsis detection chip, the method comprising:
designing a plurality of probe sequences, wherein the probe sequences comprise at lease deoxyribonucleotide sequences depicted in SEQ ID Nos. (Sequence Identifier Number) 1 to 66;
performing a probe synthesis step to synthesize a plurality of probes comprising at least the deoxyribonucleotide sequences depicted in the SEQ ID Nos. (Sequence Identifier Number) 1 to 66; and
performing a spotting step to respectively spot the probes on a matrix.

2. The fabrication method of claim 1, wherein the step of designing of the probe sequences further comprises designing a primer set corresponding to the probe sequences to amplify a specific DNA fragment of a sample of a patient, wherein the primer set is formed with DNA sequences depicted in SEQ ID NOs. 67 and 68.

3. The fabrication method of claim 1, wherein the probe synthesis step further comprises conducting an amino modification to a 5′ end of the DNA sequences depicted in the SEQ ID NOs. 1 to 66.

4. The fabrication method of claim 1, subsequent to the probe synthesis step, the method further comprises respectively dissolving the probes in deionized water to form a plurality of probe solutions.

5. The fabrication method of claim 1, wherein subsequent to the spotting step, the method further comprises performing an incubation step to maintain the matrix in a humid environment.

6. The fabrication method of claim 5, wherein subsequent to the incubation step, the method further comprises:
performing an oven-drying step to dry the matrix; and
performing a matrix cleaning step to clean the matrix.

7. The fabrication method of claim 6, wherein the matrix cleaning step further comprises:
performing a cleaning process to clean the matrix; and
performing a drying process to dry the matrix.

8. The method of claim 6, wherein subsequent to the matrix cleaning step, the method further comprises:
performing a blocking step using a blocking solution to block a matrix surface that is not spotted with the probes; and
performing another matrix cleaning step to clean the matrix.

9. The method of claim 1, wherein a spot formed by the spotting step has a radius of about 50 to 300 microns.

10. A microarray detection chip applicable for detecting whether a patient has contracted sepsis, the microarray detection chip comprising:
a plurality of probes immobilized on a matrix, and these probes are selected from the group consisting of deoxyribonucleotide sequences depicted in SEQ ID NOs. (Sequence Identifier Number) 1 to 66.

11. The detection chip of claim 10, wherein the matrix is immobilized with each of the deoxyribonucleotide sequences depicted in the SEQ ID NOs. 1 to 66.

12. The detection chip of claim 10, wherein a pathogen Clostridium septicum is identified by both probe sequences of the SEQ ID NOs. 14 and 15, a pathogen Enterococcus dispar[dyran] is identified by both probe sequences of the SEQ ID NOs. 19 and 20, a pathogen Enterococcus faecalis straink4 is identified by both probe sequences of the SEQ ID NOs.21 and 22, a pathogen Klebsiella oxytoca-16 is identified by both probe sequences of the SEQ ID NOs. 28 and 29, a pathogen Staphylococcus epidermidis is identified by both probe sequences of the SEQ ID NOs. 48 and 49, and a pathogen Staphylococcus haemolyticus is identified by both probe sequences of the SEQ ID NOs. 50 and 51.

13. The detection chip of claim 10 further comprising a plurality of quality control probes immobilized on the matrix.

14. A sepsis detection method applicable for used in detecting whether a patient has sepsis, the method comprising:
providing a microarray detection chip of claim 10;
treating a sample of the patient to extract a deoxyribonucleic acid (DNA) from the sample;
using a primer set to perform a polymerase chain reaction (PCR) on the DNA to amplify a specific fragment of the DNA to obtain a corresponding PCR product, wherein the primer set used in the PCR is formed with DNA sequences depicted in SEQ ID NOs. 67 and 68;
performing a hybridization reaction to react the PCR product with the probes on the microarray detection chip; and
performing a result analysis step on the microarray detection chip.

15. The detection method of claim 14, wherein the PCR product is labeled with a label.

16. The detection method of claim 15, wherein the label comprises a fluorescent material.

17. The detection method of claim 15, wherein the step of labeling of the PCR product comprises using at least a molecule selected from the group consisting of a primer with the label, deoxyuridine triphosphate (dUTP) with the label, and dexoyribonucleoside triphosphate (dNTP) with the label, to perform the PCR.

18. The detection method of claim 14, wherein the microarray detection chip further comprises immobilizing a plurality of quality control probes thereon.

19. The detection method of claim 14, wherein the result analysis step further comprises:
performing a scanning process on the microarray detection chip to attain a plurality of information; and
performing a data analysis process on the information.

20. The detection method of claim 14, wherein the sample comprises a blood.


SEQUENCE LISTING (68 sequences)

SEQ ID NO: 1 - DNA, 20 bp, artificial sequence
  Probe
caaatgtgaaatccccgagc

SEQ ID NO: 2 - DNA, 18 bp, artificial sequence
  Probe
ccgtgaaatcccctggct

SEQ ID NO: 3 - DNA, 27 bp, artificial sequence
  Probe
aagtctatgcataaaataccacagctc

SEQ ID NO: 4 - DNA, 17 bp, artificial sequence
  Probe
tgaaatccccgggctca

SEQ ID NO: 5 - DNA, 20 bp, artificial sequence
  Probe
ttgtgaaagtttgcggctca

SEQ ID NO: 6 - DNA, 22 bp, artificial sequence
  Probe
ctaagaccgatgtgaaatcccc

SEQ ID NO: 7 - DNA, 23 bp, artificial sequence
  Probe
taagtcagaggtgaaatcccagg

SEQ ID NO: 8 - DNA, 25 bp, artificial sequence
  Probe
tctatgcataaaataccacagctca

SEQ ID NO: 9 - DNA, 21 bp, artificial sequence
  Probe
tgcataaaataccacggctca

SEQ ID NO: 10 - DNA, 22 bp, artificial sequence
  Probe
agtcaggagtgaaaggctacgg

SEQ ID NO: 11 - DNA, 26 bp, artificial sequence
  Probe
gatgattaagtgggatgtgaaatacc

SEQ ID NO: 12 - DNA, 25 bp, artificial sequence
  Probe
tattaagtcaggggtgaaaggtttc

SEQ ID NO: 13 - DNA, 27 bp, artificial sequence
  Probe
aaggaaagttagatgttaaattttggg

SEQ ID NO: 14 - DNA, 23 bp, artificial sequence
  Probe
gcggacttttaagtgagatgtga

SEQ ID NO: 15 - DNA, 20 bp, artificial sequence
  Probe
atgtgaaatacccgggctca

SEQ ID NO: 16 - DNA, 22 bp, artificial sequence
  Probe
tctaagtcggatgtgaaagccc

SEQ ID NO: 17 - DNA, 24 bp, artificial sequence
  Probe
tgttaagtcagatgtgaaatcccc

SEQ ID NO: 18 - DNA, 23 bp, artificial sequence
  Probe
aggcggtctgttaagtcagatgt

SEQ ID NO: 19 - DNA, 23 bp, artificial sequence
  Probe
aggcggtttcttaagtctgatgt

SEQ ID NO: 20 - DNA, 23 bp, artificial sequence
  Probe
caggcggtttcttaagtctgatg

SEQ ID NO: 21 - DNA, 23 bp, artificial sequence
  Probe
aggcggtttcttaagtctgatgt

SEQ ID NO: 22 - DNA, 21 bp, artificial sequence
  Probe
gcaggcggttcttaagtctga

SEQ ID NO: 23 - DNA, 23 bp, artificial sequence
  Probe
aggcggttcttaagtctgatgtg

SEQ ID NO: 24 - DNA, 23 bp, artificial sequence
  Probe
gggtggtttgttaagttggatgt

SEQ ID NO: 25 - DNA, 26 bp, artificial sequence
  Probe
ttaagtcagtagtgaaatcttgcagc

SEQ ID NO: 26 - DNA, 24 bp, artificial sequence
  Probe
gcgggttcataagtctgatgttaa

SEQ ID NO: 27 - DNA, 23 bp, artificial sequence
  Probe
aggcggttatttaagtgaggtgt

SEQ ID NO: 28 - DNA, 21 bp, artificial sequence
  Probe
cggtctgtcaagtcggatgtg

SEQ ID NO: 29 - DNA, 21 bp, artificial sequence
  Probe
gatctggaggaataccggtgg

SEQ ID NO: 30 - DNA, 21 bp, artificial sequence
  Probe
cggtctgtcaagtcggatgtg

SEQ ID NO: 31 - DNA, 19 bp, artificial sequence
  Probe
tgtgaaaactgcgggctca

SEQ ID NO: 32 - DNA, 22 bp, artificial sequence
  Probe
aagtctgatgtgaaagccctcg

SEQ ID NO: 33 - DNA, 25 bp, artificial sequence
  Probe
aggtggtttggtaagttatctgtga

SEQ ID NO: 34 - DNA, 20 bp, artificial sequence
  Probe
gtttgtcgcgttgttcgtga

SEQ ID NO: 35 - DNA, 24 bp, artificial sequence
  Probe
tgacttttaagtcaggggtgaaat

SEQ ID NO: 36 - DNA, 25 bp, artificial sequence
  Probe
ggattgaaaagtctggtgttaaagg

SEQ ID NO: 37 - DNA, 24 bp, artificial sequence
  Probe
agacggttacttaagcaggatgtg

SEQ ID NO: 38 - DNA, 21 bp, artificial sequence
  Probe
gtcgtttgtgaaaacttgggg

SEQ ID NO: 39 - DNA, 25 bp, artificial sequence
  Probe
ggttgattgagtcagatgtgaaatc

SEQ ID NO: 40 - DNA, 25 bp, artificial sequence
  Probe
gcggttgattaagttagatgtgaaa

SEQ ID NO: 41 - DNA, 23 bp, artificial sequence
  Probe
aggcggtaaattaagtcaggtgt

SEQ ID NO: 42 - DNA, 22 bp, artificial sequence
  Probe
gtggtctttcaagtcggtggtt

SEQ ID NO: 43 - DNA, 23 bp, artificial sequence
  Probe
atgattaagcgtgacgtgaaatg

SEQ ID NO: 44 - DNA, 24 bp, artificial sequence
  Probe
caattaagtcagatgtgaaagccc

SEQ ID NO: 45 - DNA, 25 bp, artificial sequence
  Probe
aggtggttagttaagttggatgtga

SEQ ID NO: 46 - DNA, 20 bp, artificial sequence
  Probe
cgtttgtgaaaaccagcagc

SEQ ID NO: 47 - DNA, 25 bp, artificial sequence
  Probe
tttagtaagttggaagtgaaagccc

SEQ ID NO: 48 - DNA, 21 bp, artificial sequence
  Probe
agtctgatgtgaaagcccacg

SEQ ID NO: 49 - DNA, 26 bp, artificial sequence
  Probe
aactggaaaacttgagttcagaagag

SEQ ID NO: 50 - DNA, 21 bp, artificial sequence
  Probe
agtctgatgtgaaagcccacg

SEQ ID NO: 51 - DNA, 25 bp, artificial sequence
  Probe
attggaaactgtaaaacttgagtgc

SEQ ID NO: 52 - DNA, 24 bp, artificial sequence
  Probe
aggcggttctttaagtctgaagtt

SEQ ID NO: 53 - DNA, 24 bp, artificial sequence
  Probe
aggcggttagataagtctgaagtt

SEQ ID NO: 54 - DNA, 26 bp, artificial sequence
  Probe
aggcggtttaataagtctgaagttaa

SEQ ID NO: 55 - DNA, 24 bp, artificial sequence
  Probe
gcggttagaaaagtctgaagtgaa

SEQ ID NO: 56 - DNA, 23 bp, artificial sequence
  Probe
tctgaagttaaaggcattggctc

SEQ ID NO: 57 - DNA, 21 bp, artificial sequence
  Probe
tcaagtcggatgtgaagtccc

SEQ ID NO: 58 - DNA, 17 bp, artificial sequence
  Probe
tgaaatccccgggctca

SEQ ID NO: 59 - DNA, 19 bp, artificial sequence
  Probe
gtgaaatcccccgcgctta

SEQ ID NO: 60 - DNA, 21 bp, artificial sequence
  Probe
agtctgatgtgaaagcccacg

SEQ ID NO: 61 - DNA, 24 bp, artificial sequence
  Probe
aggtggtcgtttaagtctgttgtg

SEQ ID NO: 62 - DNA, 24 bp, artificial sequence
  Probe
aggcggttagataagtctgaagtt

SEQ ID NO: 63 - DNA, 19 bp, artificial sequence
  Probe
gcggactggtaagcctggt

SEQ ID NO: 64 - DNA, 20 bp, artificial sequence
  Probe
tcagatgtgaaagccctggg

SEQ ID NO: 65 - DNA, 16 bp, artificial sequence
  Probe
gaaagcccggggctca

SEQ ID NO: 66 - DNA, 18 bp, artificial sequence
  Probe
ctgatgtgaaagcccccg

SEQ ID NO: 67 - DNA, 19 bp, artificial sequence
  Primer
cggctaactctgtgccagc

SEQ ID NO: 68 - DNA, 23 bp, artificial sequence
  Primer
ggcatggactaccagggtatcta